(19) United States
(12) Patent Application Publication
(10) Pub. No.: US 2026/0013875 A1
(43) Pub. Date: Jan. 15, 2026

(54) PREPARING A TIBIA FOR RECEIVING TIBIAL IMPLANT COMPONENT OF A REPLACEMENT ANKLE

(71) Applicant: WRIGHT MEDICAL TECHNOLOGY, INC., Memphis, TN (US)

(72) Inventors: Ramon LUNA, Arlington, TN (US); Kian-Ming WONG, Lakeland, TN (US)

(73) Assignee: WRIGHT MEDICAL TECHNOLOGY, INC., Memphis, TN (US)

(21) Appl. No.: 19/333,662

(22) Filed: Sep. 19, 2025

Related U.S. Application Data
(63) Continuation of application No. 18/252,547, filed on May 11, 2023, now Pat. No. 12,440,227.
(60) Provisional application No. 63/152,996, filed on Feb. 24, 2021.

(51) Int. Cl. A61B 17/16; A61B 17/17

(52) U.S. Cl. CPC A61B 17/1682; A61B 17/1631; A61B 17/164; A61B 17/1775

(57) ABSTRACT

Disclosed is a method of preparing a tibial intramedullary canal for receiving a tibial implant such as a stem component of a total ankle replacement prosthesis where an extended cavity is prepared in the tibial intramedullary canal by pulling a tibial reamer bit in the proximal direction from the distal end of a tibia into the tibial intramedullary canal by using a flexible neck tooling introduced through the tibial intramedullary canal from a position proximal to the distal end of the tibia.

### CROSS-REFERENCE TO RELATED APPLICATIONS

This application claims priority to U.S. provisional application No. 63/152,996, filed on Feb. 24, 2021. Any and all applications for which a foreign or domestic priority claim is identified in the Application Data Sheet as filed with the present application are hereby incorporated by reference under 37 C.F.R. § 1.57.

### FIELD OF DISCLOSURE

The present disclosure relates to an ankle replacement procedure.

### BACKGROUND

An ankle joint may become severely damaged and painful due to arthritis from prior ankle surgery, bone fracture, infection, osteoarthritis, post-traumatic osteoarthritis or rheumatoid arthritis, for example. Options for treating the injured ankle have included anti-inflammatory and pain medications, braces, physical therapy, amputation, joint arthrodesis, and total ankle replacement.

Current ankle joint replacement options include preparing the distal end of the tibia by drilling through the calcaneus and the talus from the bottom of the foot to access the distal end of the tibia to ream the tibial intramedullary canal. Such approaches require an additional incision in the heel. The patient's recovery time can be extended and can delay the weight-bearing time after the surgery.

A recent improved ankle joint replacement procedure involves approaching the ankle joint space with a broach from the anterior side and preparing the intramedullary canal of the tibia manually.

### SUMMARY

Disclosed is a method of preparing an intramedullary canal in a tibia for receiving a tibial implant. In some embodiments, the method comprises:
(a) forming an ankle joint space by resecting the distal end of a tibia;
(b) forming a cavity for a bone interface feature into the distal end of the tibia, wherein the cavity has a proximal end that is closed within the tibia and an opening at the distal end of the tibia;
(c) drilling a hole into the tibia using a flexible reamer fitted with a pilot cutting bit from a location that is proximal with respect to the cavity formed in the tibia until the flexible reamer exits into the cavity at a location that is at the proximal end of the cavity;
(d) inserting the flexible reamer further into the cavity until the pilot cutting bit is within the ankle joint space;
(e) slipping a stem reamer guide over the pilot cutting bit and inserting the stem reamer guide into the cavity's opening at the distal end of the tibia;
(f) adjusting the stem reamer guide in anterior-posterior direction to align to flexible reamer's trajectory while bet centering in coronal-sagittal direction depending on the insertion point direction;
(g) replacing the pilot cutting bit with a tibial reamer bit that is configured for reaming in the proximal direction; and
(h) forming an extended cavity by pulling the tibial reamer bit through the stem reamer guide in the proximal direction and reaming the proximal end of the cavity by pulling the flexible reamer in the proximal direction.

According to another aspect of the present disclosure, the method comprises:
(a) forming an ankle joint space by resecting the distal end of a tibia;
(b) forming a cavity for a bone interface feature into the distal end of the tibia, wherein the cavity has a proximal end that is closed within the tibia and an opening at the distal end of the tibia;
(c) adjusting the alignment assembly for stem reamer guide placement in anterior-posterior direction to prepare a hole into the tibia;
(d) inserting a stem reamer guide into the cavity's opening at the distal end of the tibia;
(e) drilling a hole into the tibia using a flexible reamer fitted with a pilot cutting bit from a location that is proximal with respect to the cavity while centering in coronal-sagittal direction depending on the insertion point direction;
(f) inserting the flexible reamer further into the cavity until the pilot cutting bit extends through the stem reamer guide and is within the ankle joint space;
(g) replacing the pilot cutting bit with a tibial reamer bit that is configured for reaming in the proximal direction; and
(h) forming an extended cavity by pulling the tibial reamer bit through the stem reamer guide in the proximal direction and reaming the proximal end of the cavity by pulling the flexible reamer in the proximal direction.

### BRIEF DESCRIPTION OF THE DRAWINGS

The inventive concepts of the present disclosure will be described in more detail in conjunction with the following drawing figures. The structures in the drawing figures are illustrated schematically and are not intended to show actual dimensions.

FIG. 1 is a flowchart representation of a method according to an embodiment of the present disclosure.

FIG. 2 is a flowchart representation of a method according to another embodiment of the present disclosure.

FIG. 3A illustrates one example of tibial and talar resections having been made in accordance with some embodiments.

FIG. 3B illustrates one example of an ankle joint space formed by the tibia and ankle resections shown in FIG. 3A.

FIG. 4 illustrates the anterior view of the step of attaching a stem reamer bit to the head of the flexible reamer according to an aspect of the present disclosure.

FIG. 5 illustrates lateral view of the step of drilling a hole in the tibia using a flexible reamer according to an aspect of the present disclosure.

FIG. 6A is a top-down view illustration of a guide for a 90° reamer for forming a hole/cavity in the distal end of a tibia for receiving a stem reamer guide.

FIG. 6B is an anterior view illustration of the guide of FIG. 6A.

FIG. 6C is a cross-sectional view of the guide shown in FIGS. 6A-6B, where the section is taken through the section line 6C-6C shown in FIG. 6B.

FIG. 6D is an anterior view of an alignment jig that can be used to align the guide of FIG. 6A.

FIG. 6E is a medial view of the alignment jig of FIG. 6D

FIG. 7A is an illustration of a pilot cutting bit for a flexible reamer.

FIG. 7B is an illustration of a bottom view of the pilot cutting bit of FIG. 7A.

FIG. 7C is an illustration of a cross-sectional view of the pilot cutting bit of FIGS. 7A-7B taken through the section line 7C-7C shown in FIG. 7B.

FIG. 8A is an illustration of a stem reamer guide.

FIG. 8B is a side view illustration of the stem reamer guide of FIG. 8A.

FIG. 8C is a cross-sectional view illustration of the stem reamer guide, where the section is taken through the section line 8C-8C shown in FIG. 8B.

FIGS. 9A and 9B are a side view and a detailed side view illustrations, respectively, of an example of a flexible reamer shaft that can be used with the method disclosed herein.

FIG. 9C is an end view illustration the flexible reamer shaft.

FIG. 10A is a side view illustration of a tibial stem reamer bit.

FIG. 10B is an end view illustration of the tibial stem reamer bit of FIG. 10A.

FIG. 10C is a cross-sectional view illustration of the tibial stem reamer bit, where the section is taken through the section line 10C-10C shown in FIG. 10B.

FIG. 11A is an end view illustration of a tibial reamer bit extender.

FIG. 11B is a side view illustration of the tibial reamer bit extender of FIG. 11A.

FIG. 11C is a cross-sectional view illustration of the tibial reamer bit extender, where the section is taken through the section line 11C-11C shown in FIG. 11B.

FIG. 12 illustrates a tibial portion of an example total ankle prosthesis having a tibia stem component.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Unless otherwise expressly stated, it is in no way intended that any method set forth herein be construed as requiring that its steps be performed in a specific order, nor that with any apparatus, specific orientations be required, unless specified as such. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

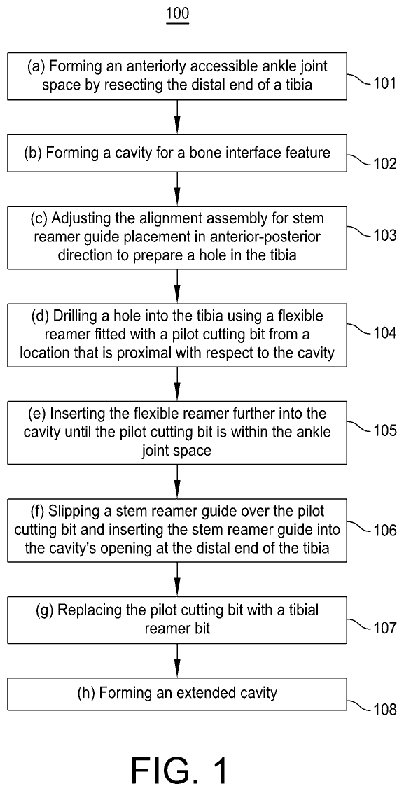

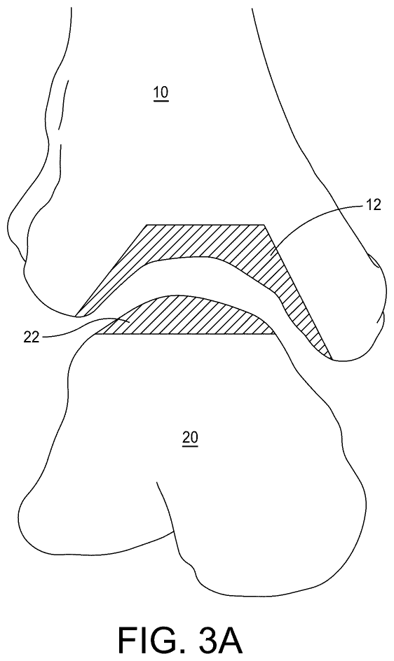

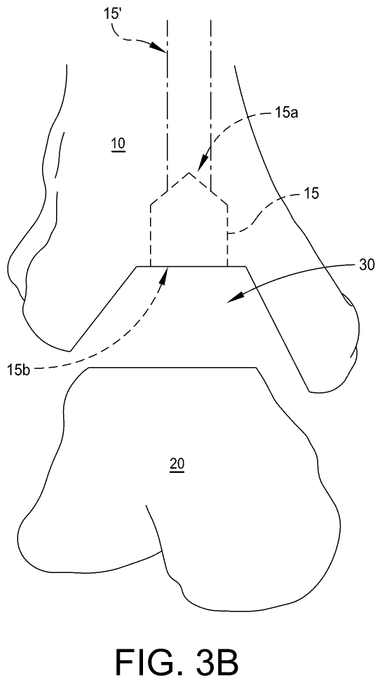

Referring to FIG. 1, an embodiment of a method for reaming a cavity in a tibia for receiving a tibial stem component of a total ankle replacement prosthesis according to the present disclosure is represented by the flowchart 100. FIGS. 3A-3B are illustrations of a tibia 10 and a talus 20 of an exemplary ankle joint to help with the description.

The method comprises a step (a) of forming an ankle joint space 30 by resecting a portion 12 of the distal end of a tibia 10 by making a box cut as shown in FIG. 3A. If a larger ankle joint space is necessary, a superior portion 22 of the associated talus 20 can also be resected at this time. (Box 101 in the flowchart 100). In some embodiments, the ankle joint space 30 thus formed is anteriorly accessible. In some other embodiments, the ankle joint space 30 can be formed to be accessible from other direction if needed.

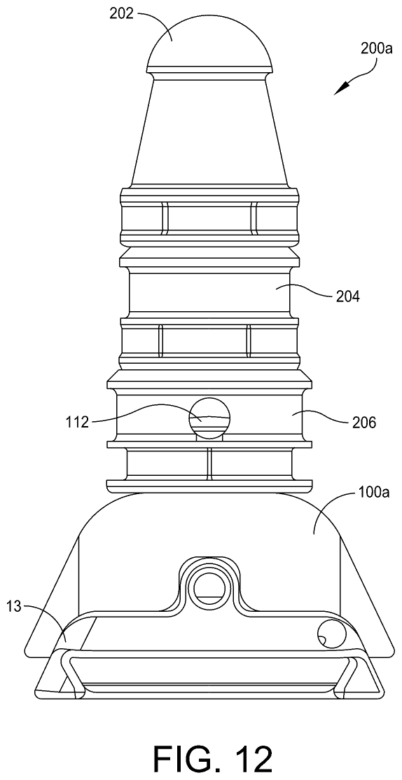

Next referring to FIG. 3B, in step (b), a cavity 15 is formed into the distal end of the tibia 10 for receiving a bone interface feature such as a tibial stem component 200a (see FIG. 12). (Box 102). The cavity 15 formed in the tibia 10 has a proximal end 15a that is closed within the tibia and an opening 15b at the distal end of the tibia 10.

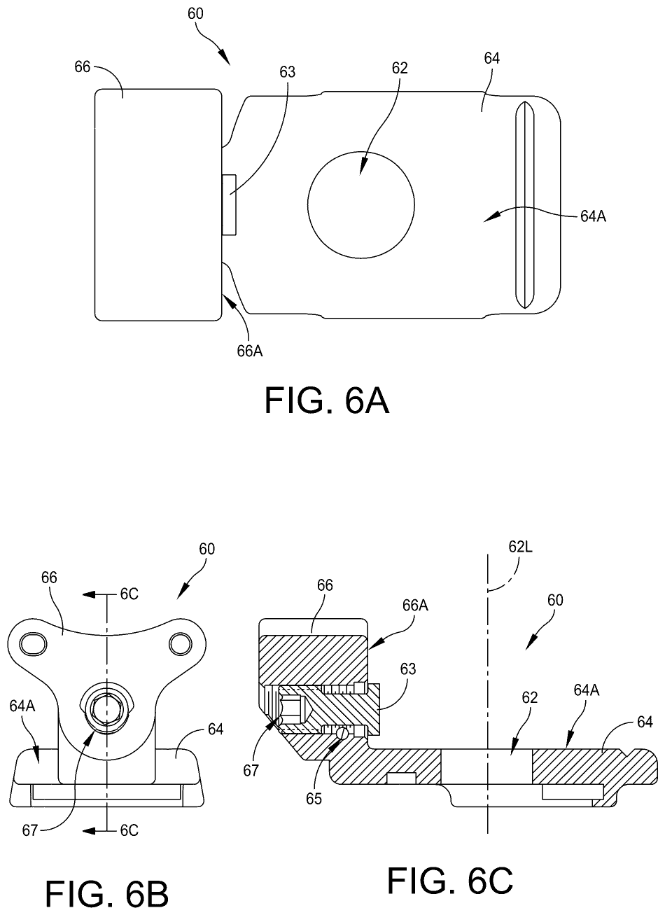

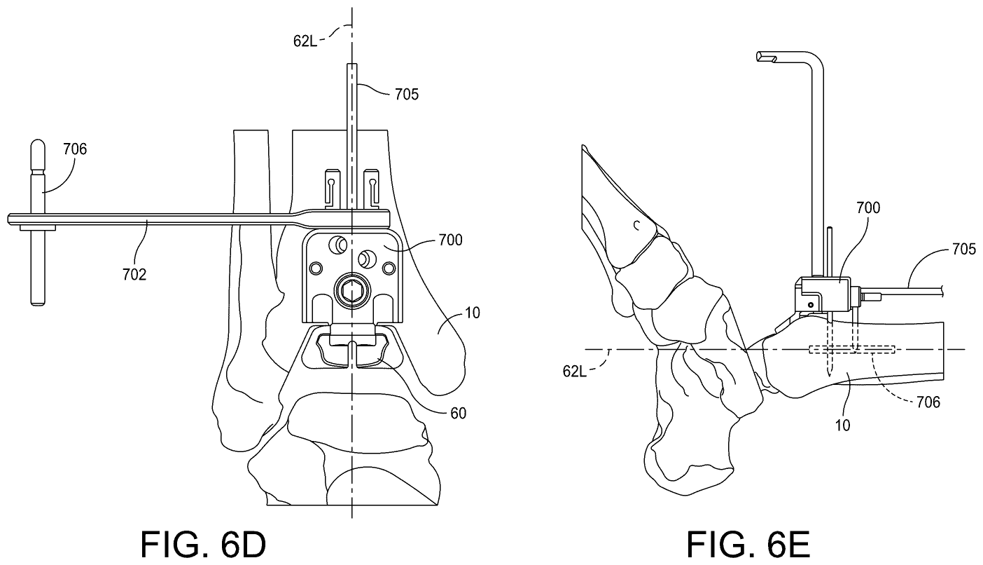

The cavity 15 can be formed using a 90° reamer tool that has an angled neck that is configured to drive a reamer bit that is oriented 90° with respect to the handle of the reamer tool. The 90° reamer tool can ream a hole into a bone in a direction that is orthogonal to the handle of the reamer tool. Such tool can be inserted into the ankle joint space 30, defined by the box cut shown in FIG. 3A, and ream the cavity 15 into the distal end of the tibia 10. To aid in reaming the cavity 15 in the desired location on the resected distal surface of the tibia, a 90° reaming alignment guide can be used. An example of such 90° reaming alignment guide 60 is shown in FIGS. 6A-6C.

The 90° reaming alignment guide 60 comprises a substantially flat body 64 and a reamer guide hole 62 provided in the flat body 64. The flat body has a top surface 64A that is intended to butt up against the resected distal surface of the tibia when aligning the guide hole 62 on the tibia. The guide hole 62 will guide the reamer bit of the 90° reamer tool for reaming the cavity 15. The alignment guide 60 further comprises a referencing tab 66 that extends orthogonal to the flat body 64 and helps align the position of the guide hole 62 with respect to the distal end of the tibia. The referencing tab 66 has a surface 66A that is orthogonal to the top surface 64A and forms a right angle corner with the top surface 64A as shown in FIG. 6C. The referencing tab 66 comprises an alignment platen 63 that is threadedly engaged in a threaded hole 67. In use, the flat body 64 of the guide is inserted into the ankle joint space 30 from anterior side and the top surface 64A of the alignment guide 60 is placed against the resected distal surface of the tibia with the alignment platen 63 contacting the anterior side of the distal end of the tibia. Then, the alignment of the guide hole 62 in the anterior-posterior direction with respect to the resected distal surface of the tibia can be adjusted by threading or unthreading the alignment platen 63. By unthreading the alignment platen 63 so that the platen 63 protrudes out of the threaded hole 67, the platen 63 will push against the anterior side of the distal end of the tibia and cause the rest of the guide 60 including the guide hole 62 to move in the anterior direction. By threading the alignment platen 63 into the threaded hole 67, the guide 60 including the guide hole 62 is moved in the posterior direction.

Referring to FIGS. 6D and 6E, an alignment jig assembly, such as the example alignment jig assembly 700, can be used to assist in aligning the alignment guide 60 in a proper place to ream the cavity 15. FIG. 6D shows an anterior view of the alignment assembly 700 that is attached to the guide 60 and the combined assembly is secured to the distal end of the tibia 10 from the anterior side. The alignment assembly 700 comprises an Anterior-Posterior alignment rod 706 and a Medial-Lateral (i.e. coronal-sagittal) alignment rod 705.

As can be seen in the anterior view in FIG. 6D, the Anterior-Posterior alignment rod 706 is attached to the alignment assembly 700 by an alignment wing 702. The alignment assembly 700 is configured so that the Anterior-Posterior alignment rod 706 extends parallel to the longitudinal axis 62L of the guide hole 62 of the guide 60 so that the Anterior-Posterior alignment rod 706 can be used to align the longitudinal axis 62L with the tibia 10 in the Anterior-Posterior direction. The longitudinal axis 62L represents the direction of reaming that would be performed using the guide hole 62 as a guide for the 90° degree reamer tool discussed above. The alignment can be checked simply visually or with the aid of a fluoroscope. As can be seen in the medial view in FIG. 6E, when the Anterior-Posterior alignment rod 706 is aligned with the tibia 10, the longitudinal axis 62L of the guide hole 62 is also aligned with the tibia 10 in the Anterior-Posterior direction. Thus, while viewing from the medial or lateral side, the surgeon can check the Anterior-Posterior alignment of the guide hole 62 with respect to the tibia 10 and adjust the position of the alignment guide 60 as necessary ensuring that the reaming through the guide hole 62 will advance into the tibia 10 in the direction desired by the surgeon.

Similarly, the alignment assembly 700 is configured so that the Medial-Lateral alignment rod 705 extends parallel to the longitudinal axis 62L of the guide hole 62 so that the Medial-Lateral alignment rod 705 can be used to align the longitudinal axis 62L with the tibial 10 in the Medial-Lateral direction. As can be seen in the medial view in FIG. 6E, the Medial-Lateral alignment rod 705 extends from the aignment assembly 700 and is positioned above the tibia 10 on the anterior side of the tibia 10. The alignment in the Medial-Lateral direction can be checked simply visually or with the aid of a fluoroscope viewing from the anterior side as shown in FIG. 6D. When the Medial-Lateral alignment rod 705 is aligned with the tibia 10, the longitudinal axis 62L of the guide hole 62 is also aligned with the tibia 10 in the Medial-Lateral direction. Thus, while viewing from the anterior side as shown in FIG. 6D, the surgeon can check the Medial-Lateral alignment of the guide hole 62 with respect to the tibia 10 and adjust the position of the alignment guide 60 if necessary.

Referring to FIG. 6D, the Anterior-Posterior alignment rod 706 is positioned to the lateral side of the tibia 10. The Anterior-Posterior alignment rod 706 is also parallel to the longitudinal axis of the guide hole 62. When viewing under a fluoroscope from the medial side as shown in FIG. 6E, the Anterior-Posterior alignment rod 706 is visible through the tibia 10. Thus, the longitudinal axis of the guide hole 62 can be aligned to be in a desired position within the intramedullary canal of the tibia in the Anterior-Posterior direction under a fluoroscope. Thus, using the alignment assembly 700, the guide hole 62 in the 90° reamer alignment guide 60 is aligned in both the Medial-Lateral direction as well as the Anterior-Posterior direction.

Once the alignment guide 60 is positioned with the guide hole 62 in desired alignment, the alignment guide 60 can be secured in place, with appropriate pins for example, and a 90° reamer is used to ream the cavity 15 through the guide hole 62 of the alignment guide 60 from the ankle joint space 30.

Next, in step (c), using the alignment assembly 700, the position of the stem reamer guide 70 is adjusted in Anterior-Posterior direction to align to flexible reamer's trajectory while centering in Medial-Lateral direction depending on the trajectory of the pilot cutting bit 51 drilling downward from the hole 17. (Box 103). This adjustment using the alignment assembly 700 can be performed under a fluoroscope.

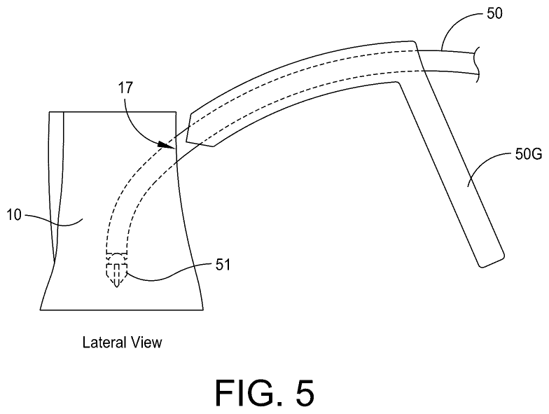

Next, referring to FIG. 5, in step (d), a hole 17 is formed into the tibia 10 using a flexible reamer 50 fitted with a pilot cutting bit 51 from a location that is proximal with respect to the cavity 15 until the flexible reamer 50 exits into the cavity 15 at a location that is at the proximal end 15a of the cavity 15. (Box 104). FIG. 5 is a lateral view of a tibia 10 showing the arrangement where the hole 17 is drilled on the anterior side of the tibia 10. The hole 17 can be drilled in the anterior, medial, or posterior side of the tibia. The lateral side would not be feasible because of the fibula. In some embodiments, the preferred placement of the hole 17 is on the anterior side. Drilling of the hole 17 can be achieved with a cannulated drill guide 50G but it could be from straight to a curve drill guide or adjustable drill guide to control the path of the flexible reamer.

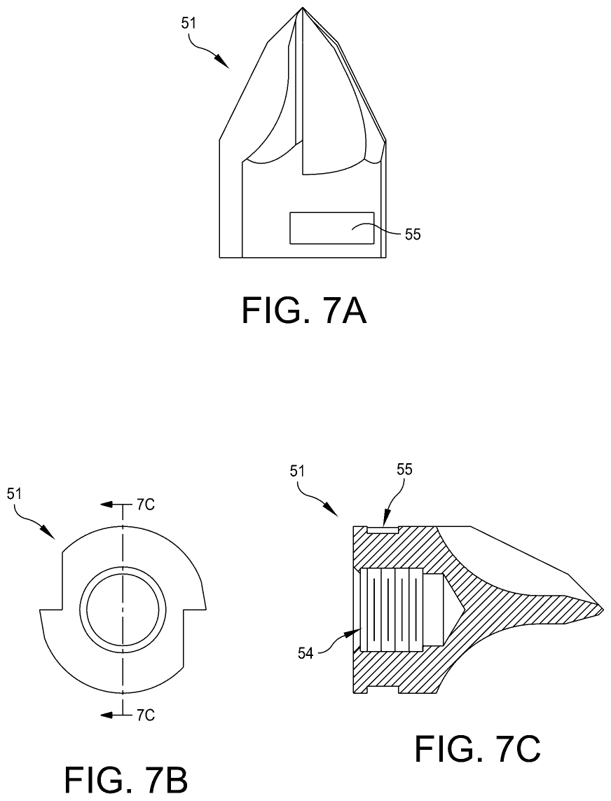

The pilot cutting bit 51 can be any tool bit that is suitable for forming the hole 17 into the tibial 10. Some examples of such cutting bit are a standard reamer bit, a drill bit, a broach, etc. An example of a reamer bit as the pilot cutting bit 51 is illustrated in FIGS. 7A-7C. The pilot reamer bit 51 can comprise a threaded hole 54 for attaching to the flexible reamer 50.

Next, in step (e), the flexible reamer 50 is inserted further into the cavity 15 until the pilot cutting bit 51 is within the ankle joint space 30. (Box 105).

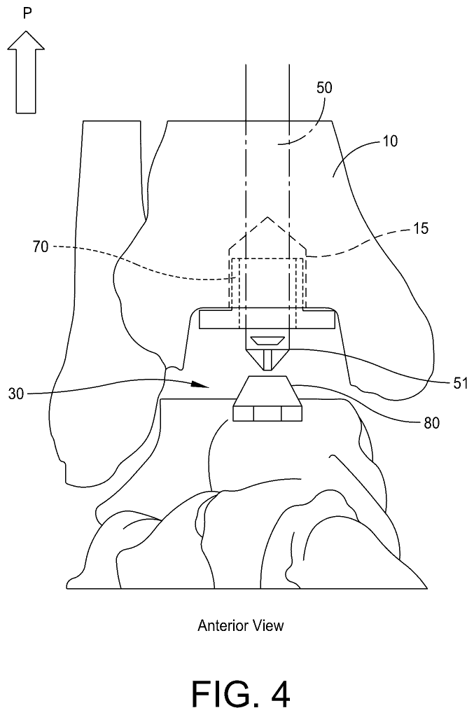

Next, referring to FIG. 4, in step (f), a stem reamer guide 70 is slipped over the pilot cutting bit 51 and inserted into the cavity's opening 15b at the distal end of the tibia 10. (Box 106).

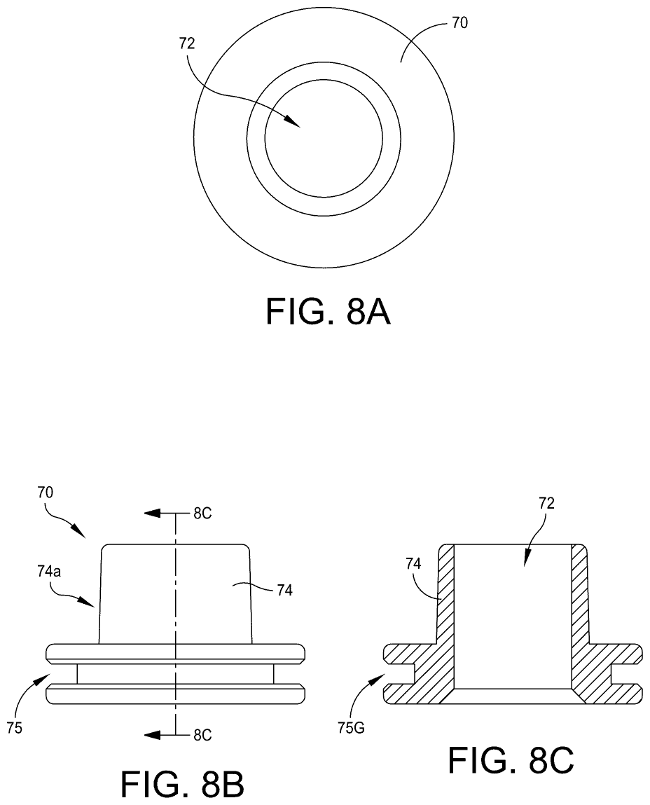

In some embodiments, the stem reamer guide 70 can be a bushing-like structure shown in FIGS. 8A-8C. The stem reamer guide 70 comprises a first portion 74 configured for inserting into the cavity's opening 15b and a second portion 75 that is configured for receiving a tool for assisting insertion into the cavity opening 15b. For example, the surface 74a of the first portion 74 can be tapered to form a press fit with the cavity opening's bone surface. In some embodiments, the first portion 74 can be configured with any other mechanism that will help keep the stem reamer guide 70 in place in the cavity's opening 15b. Some examples of such mechanism are leaf spring, tabs, straight/texture, grooves, etc. The second portion 75 extends outward radially as shown and can be configured with a groove 75G for receiving the tool for assisting the insertion into the cavity. The tool can be a wrench that fits into the groove in the second portion 75 to impact the stem reamer guide 70 into the cavity opening 15b. The stem reamer guide 70 also comprises a guide hole 72 extending there through for guiding a tibia stem reamer bit 80 disclosed herein, in the subsequent reaming procedure described herein. Multiple sizes of the stem reamer guide 70 can be provided in a surgical kit to accommodate different tibial stem implant sizes.

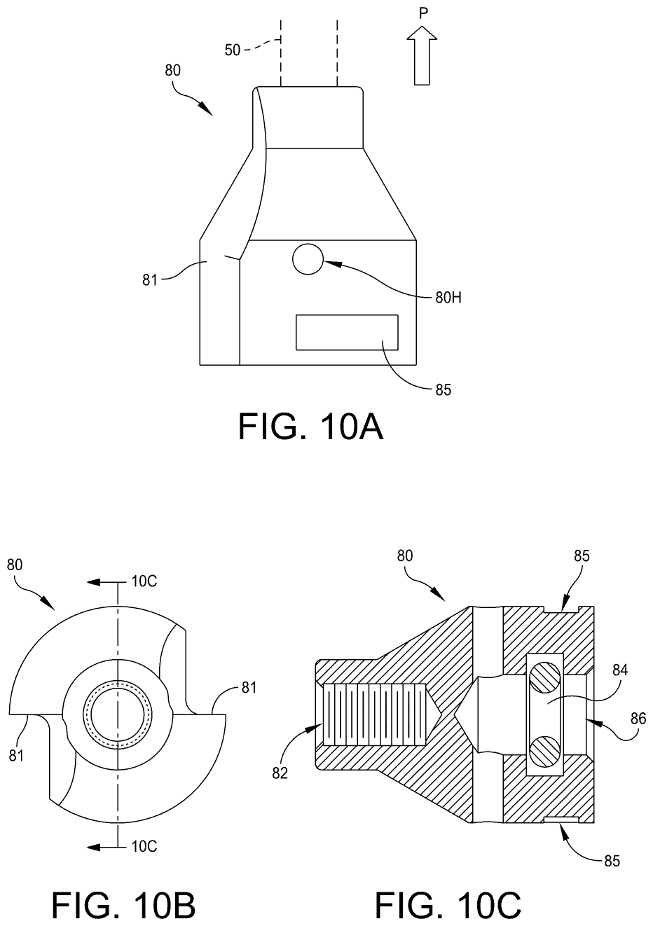

Next, in step (g), the pilot cutting bit 51 is replaced with a tibial reamer bit 80 (see FIGS. 10A-10C). (box 107). FIG. 10A shows the tibial reamer bit 80 attached to the end of the flexible reamer 50. The tibial reamer bit 80 is a reamer bit that is configured and adapted for reaming when pulled by the flexible reamer 50 in the proximal direction noted by the arrow P shown in FIG. 10A.

Next, in step (h), by pulling on the flexible reamer 50 and, thus, the tibial reamer bit 80 through the stem reamer guide 70 in the proximal direction P, the cavity 15 is extended in the proximal direction by reaming the intramedullary canal of the tibia. (Box 108).

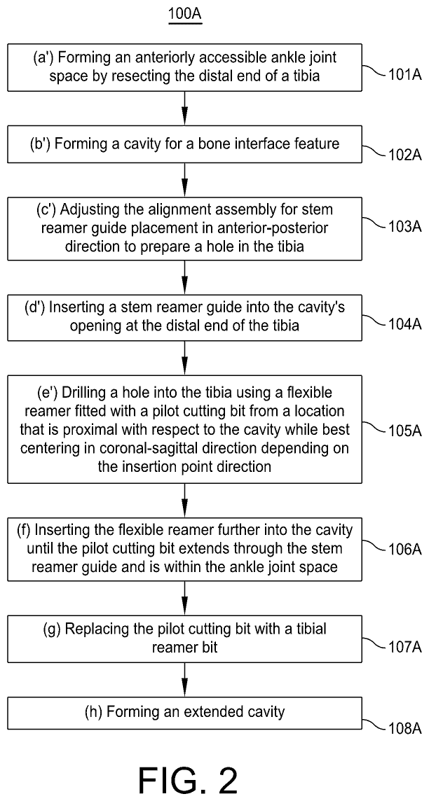

In some embodiments, the order of the steps (d)-(f) can be different. This alternate procedural steps are illustrated by flowchart 100A in FIG. 2. In this alternate example, the steps (a′)-(c′) illustrated in boxes 101A-103A are the same as steps (a)-(c) of flowchart 100. Then, (d′) the flexible reamer 50 is inserted further into the cavity 15 until the pilot cutting bit 51 is within the ankle joint space. (Box 104A).

Next, in step (e′), a hole 17 is formed into the tibia 10 using the flexible reamer 50 fitted with the pilot cutting bit 51 from a location that is proximal with respect to the cavity 15 until the flexible reamer 50 exits into the cavity 15 at a location that is at the proximal end 15a of the cavity 15. (Box 105A).

Next, in step (f′), the flexible reamer 50 is inserted further into the cavity 15 until the pilot cutting bit 51 extends through the guide hole 72 of the stem reamer guide 70 and into the ankle joint space 30. (Box 106A).

Then, the procedure is continued with the steps (g′)-(h′), (boxes 107A-108A). that are the same as the steps (g)-(h) in flowchart 100.

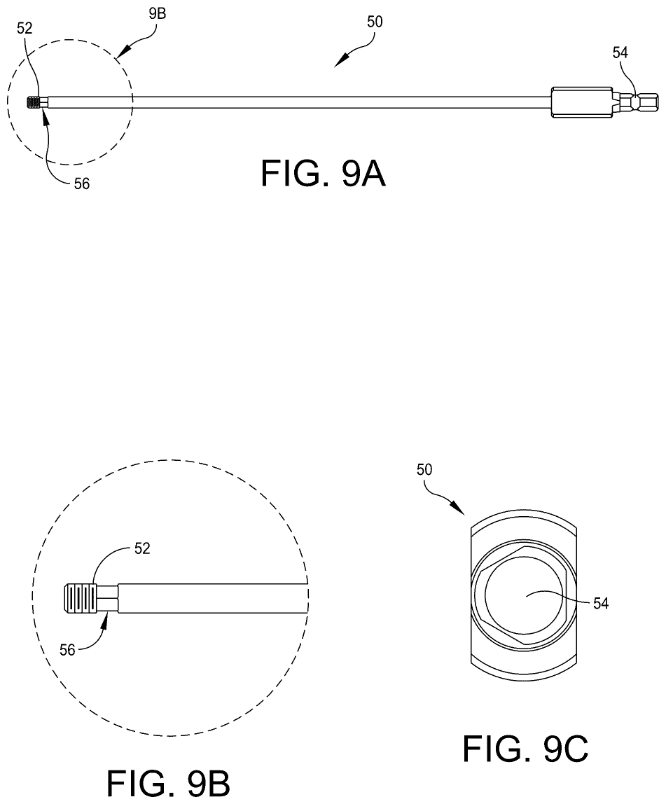

The tibial reamer bit 80 comprises one or more cutting blades 81. As shown in the cross-sectional view in FIG. 10C, the leading end of the tibial reamer bit 80 can be provided with a threaded hole 82 for attaching to the flexible reamer 50. An example of a flexible reamer 50 is shown in FIGS. 9A-9B. The flexible reamer 50 comprises a tip 52 that is configured to be removably attached to a reamer bit such as the pilot cutting bit 51 and the tibial reamer bit 80. In one embodiment, the tip 52 is threaded to mate with the threaded hole 54 of the pilot cutting bit 51 or the threaded hole 82 of the second reamer bit 80. To facilitate threaded attaching and detaching of the reamer bits 51, 80 to the flexible reamer, the reamer bits are configured to be able to be turned with a tool such as a wrench or a plier. In the illustrated examples of the reamer bits 51 and 80, the reamer bits comprise flat surfaces 55, 85, respectively, so that a wrench can be used to assist in threading and unthreading the reamer bits onto the tip 52 of the flexible reamer 50. The flexible reamer 50, in turn, comprises similar feature, such as a hex flat-sided feature near the threaded tip 52 so that a wrench can be used to provide counter-torque when threading and unthreading the reamer bits onto the tip 52. The tibial reamer bit 80 can also comprise a hole 80H for receiving a removal tool that can be used to detach the reamer bit 80 from the flexible reamer 50.

Once the tibial reamer bit 80 is attached to the end of the flexible reamer 50, in step (h), the proximal end of the cavity 15 is reamed by pulling the tibial reamer bit 80 through the guide hole 72 in the stem reamer guide 70 in the proximal direction P (see FIG. 10A) to form an extended cavity 15′ (see FIG. 3B) by pulling the flexible reamer 50 in the proximal direction P. (Box 108).

The extended cavity 15′ will be sized for receiving a tibial stem component. The flexible reamer 50 can be pulled in the proximal direction P as far as necessary. Generally, the diameter of the extended cavity 15′ will be sized to match the size of the intended tibial stem component. The tibial reamer bit 80 can be provided in various diameter sizes to accomplish this.

Once the extended cavity 15′ is reamed to a desired length, a bone interface feature can be inserted into the extended cavity 15′. An example of a bone interface feature is a tibia stem component 202 of an ankle replacement prosthesis. An example of such tibia stem component 202 is shown in FIG. 12. FIG. 12 illustrates one embodiment of a tibial portion 200a of a total ankle prosthesis having a tibial tray revision implant 100a coupled thereto. The tibial portion 200a includes a tibia stem 202 having one or more stem components 204, 206. The tibial revision implant 100a is coupled to the bottom stem component 206. For example, in some embodiments, the head 112 is inserted into a hole formed in a bottom surface of the tibia stem 202 (not shown).

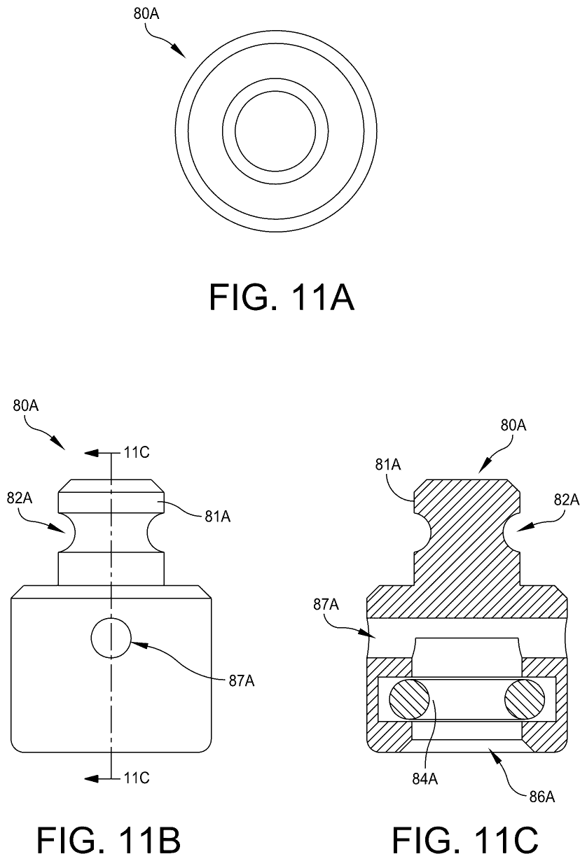

Referring to FIGS. 11A-11B, an extender 80A for tibial reamer bit 80 is disclosed. For example, if a tibial reamer bit 80 is 10 mm in length, and the extended cavity 15′ to be reamed is to be 20 mm deep from the distal surface 73 of the stem reamer guide 70, an extender 80A that is at least 10 mm long would be snapped on to the tibial reamer bit 80. The extender 80A does not have any cutting blades because it is not necessary. The extender 80A serves to lengthen the distal end of the tibial reamer bit 80 so that as the tibial reamer bit 80 is pulled further into the tibia through the guide hole 72 in the stem reamer guide 70, the extender 80A portion of the now extended reamer 80 will remain within the guide hole 72 and maintain the tibial reamer bit 80 in a proper attitude.

The extender 80A is attached to the tibial reamer bit 80 by inserting the leading end 81A of the extender 80A into the extension recess 86 in the tibial reamer bit 80. A snap ring 84 is provided inside the extension recess 86 to facilitate snap-fit engagement with the extender 80A. The leading end 81A of the extender 80A comprises an annular groove 82A for receiving the snap ring 84 when the leading end 81A is inserted into the extension recess 86. The extender 80A is provided with its own extension recess 86A and a snap ring 84A situated within the extension recess 86A to receive and engage another extender 80A by a snap-fit engagement to further extend the tibial reamer bit 80 if necessary.

To remove the extender 80A from the tibial reamer bit 80, a removal rod or a pin is inserted through the hole 87 (see FIGS. 10A, 10C) provided in the body of the tibial reamer bit 80 and push the extender 80A out. As can be seen in the cross-sectional view of the tibial reamer bit 80 in FIG. 10C, the hole 87 intersects the blind end of the extension recess 86. When the extender 80A is snap-fitted and fully seated in the extension recess 86, the leading end 81A of the extender is positioned within the intersection of the extension recess 86 and the hole 87. When the removal rod or a pin is inserted into the hole 87 and all the way through the reamer 80, along the way, the removal rod engages the chamfered edge 81C of the leading end 81A and urges the extender 80A out of the extension recess 86. The force is sufficient enough to overcome the holding spring force of the snap ring 84.

When two extenders 80A are snapped together, i.e., a trailing extender is snapped into the extension recess 86A of the leading extender, removing the trailing extender from the leading extender works the same way. A removing rod is inserted into the hole 87A (see FIGS. 11B, 11C) all the way through the leading extender 80A and the removing rod engages the chamfered edge 81C of the leading end 81A of the trailing extender and urge it out of the extension recess 86A of the leading extender.

In some embodiments, after the step (h), the disclosed method further comprises attaching a first tibial reamer bit extender 80A to a distal end of the tibial reamer bit 80 and continue reaming the extended cavity 15′ by pulling the flexible reamer 50 in the proximal direction.

In some embodiments, the disclosed method can further comprise the step (i) of attaching a second tibial reamer bit extender 80A to a distal end of the first reaming guide extender 80A and continue reaming the proximal end of the cavity 15 by pulling the flexible reamer 50 in the proximal direction.

It will be understood that the foregoing description is of exemplary embodiments of this invention, and that the invention is not limited to the specific forms shown. Modifications may be made in the design and arrangement of the elements without departing from the scope of the invention.

We claim:

1. An alignment guide for guiding a 90-degree reamer to form a cavity in a distal end of a tibia, the alignment guide comprising:
a substantially flat body having a top surface configured to butt against a resected distal surface of the tibia;
a reamer guide hole extending through the flat body and defining a longitudinal axis; and
a referencing tab outwardly extending in orthogonal relation to the flat body, having a surface that forms a right-angle corner with the top surface, the referencing tab defining a threaded hole adapted for receiving a threaded alignment platen that, when threaded and unthreaded, adjusts a position of the reamer guide hole in an anterior-posterior direction relative to the tibia.

2. The alignment guide of claim 1, wherein the flat body is sized and configured for insertion into an anteriorly accessible ankle joint space.

3. The alignment guide of claim 1, wherein the threaded alignment platen is configured to contact an anterior side of the distal end of the tibia so as to relocate the reamer guide hole anteriorly when unthreaded and posteriorly when threaded.

4. The alignment guide of claim 1, wherein the longitudinal axis of the reamer guide hole defines a direction of reaming orthogonal to a handle of a 90-degree reamer tool.

5. The alignment guide of claim 1, further defining one or more pin-receiving holes arranged to receive a pin so as to secure the alignment guide to the tibia during reaming.

6. The alignment guide of claim 1, wherein the referencing tab is configured to align the reamer guide hole with respect to an edge of the distal end of the tibia.

7. The alignment guide of claim 1, wherein the flat body and the referencing tab are observable for alignment under fluoroscopy.

8. An alignment assembly configured to align the reamer guide hole of the alignment guide of claim 1, the alignment assembly comprising:
an anterior-posterior alignment rod;
a medial-lateral alignment rod; and
wherein the alignment assembly is coupled to the alignment guide such that both the anterior-posterior and medial-lateral alignment rods extend parallel to the longitudinal axis of the reamer guide hole to enable concurrent anterior-posterior and medial-lateral alignment relative to the tibia.

9. The alignment assembly of claim 8, wherein the anterior-posterior alignment rod is positioned lateral to the tibia and is visible through the tibia under fluoroscopy when viewed from a medial side.

10. The alignment assembly of claim 8, wherein the medial-lateral alignment rod is positioned above the tibia on an anterior side to enable anterior visualization of medial-lateral alignment.

11. The alignment assembly of claim 8, wherein the anterior-posterior alignment rod is carried by an alignment wing.

12. A stem reamer guide comprising:
a first portion configured to be received in an opening of a cavity at the distal end of a tibia;
a second portion extending radially outwardly of the first portion and including a tool-engaging groove; and
a guide hole defined through the first portion and the second portion adapted and arranged to guide a tibial stem reamer bit, wherein the first portion includes a tapered outer surface configured to form a press-fit with bone at the cavity opening.

13. The stem reamer guide of claim 12, wherein the first portion comprises at least one of a leaf spring, tab, surface texture, and grooves to retain the stem reamer guide within the cavity opening.

14. The stem reamer guide of claim 12, wherein the tool-engaging groove is configured to receive an insertion tool adapted for impacting the stem reamer guide into the cavity opening.

15. The stem reamer guide of claim 12, wherein a plurality of stem reamer guides of different sizes is provided to accommodate different tibial stem implant sizes.

16. A flexible reamer system comprising:
a flexible reamer having a threaded tip and a flat-side located adjacent to the threaded tip;
a pilot cutting bit defining a threaded hole so as to threadably couple to the threaded tip; and
a tibial reamer bit defining a threaded hole arranged so as to threadably couple to the threaded tip, the tibial reamer bit including one or more cutting blades and being configured for reaming when pulled in a proximal direction.

17. The system of claim 16, wherein at least one of the pilot cutting bit and the tibial reamer bit includes flats circumferentially arranged receive a tool to facilitate threading and unthreading relative to the threaded tip.

18. The system of claim 16, wherein the tibial reamer bit defines a hole configured to receive a removal tool to facilitate detachment from the flexible reamer.

19. The system of claim 16, further including a drill guide configured to control a path of the flexible reamer from a proximal tibial location.

20. A reamer bit extender system comprising:
a tibial reamer bit having an extension recess at a distal end, a snap ring disposed within the extension recess, and a transverse hole intersecting a blind end of the extension recess; and
an extender having a leading end with an annular groove configured to snap-fit with the snap ring when the leading end is received in the extension recess, the extender further including an extension recess and snap ring to receive a trailing extender by snap-fit engagement.

21. The system of claim 20, wherein the tibial reamer bit includes a chamfered edge at the leading end of the extender arranged so as to be received in the extension recess such that insertion of a removal rod through the transverse hole engages the chamfered edge to urge the extender out of the extension recess.

22. The system of claim 20, wherein the extender defines a transverse hole intersecting a blind end of an extension recess to enable removal of a snapped-in trailing extender.

23. The system of claim 20, wherein the extender is configured to lengthen the tibial reamer bit such that a distal portion of the reamer assembly remains within a guide hole, defined in a stem reamer guide, during proximal pulling.

24. The system of claim 20, wherein a plurality of extenders is stackable to obtain a predetermined effective length of the reamer assembly.

25. A method of aligning a 90-degree reamer relative to a tibia, comprising:
inserting an alignment guide having a flat body that defines a reamer guide hole into an anteriorly accessible ankle joint space;
placing a top surface of the flat body against a resected distal surface of the tibia;
adjusting a threaded alignment platen on a referencing tab of the alignment guide to move the reamer guide hole at least one of anteriorly or posteriorly;
coupling an alignment assembly to the alignment guide such that anterior-posterior and medial-lateral alignment rods of the assembly extend parallel to a longitudinal axis of the reamer guide hole;
aligning the rods with the tibia in both anterior-posterior and medial-lateral directions under visual observation or fluoroscopy;
securing the alignment guide to the tibia; and
reaming through the reamer guide hole with a 90-degree reamer to form a cavity for a bone interface feature.

26. The method of claim 25, wherein adjusting the threaded alignment platen includes unthreading the platen so as to be configured to push against an anterior side of the distal end of the tibia to move the reamer guide hole anteriorly, and threading the platen to move the reamer guide hole posteriorly.

27. The method of claim 25, wherein aligning the anterior-posterior alignment rod comprises positioning the rod lateral to the tibia and viewing the rod through the tibia under fluoroscopy from a medial side.

28. The method of claim 25, wherein aligning the medial-lateral alignment rod comprises viewing the rod from an anterior side of the tibia.

29. A method of coupling and decoupling reamer bits to a flexible reamer, comprising: threading a pilot cutting bit onto a threaded tip of a flexible reamer while applying torque to wrench flats on the pilot cutting bit and counter-torque to a flat-sided feature on the flexible reamer; unthreading the pilot cutting bit; threading a tibial reamer bit onto the threaded tip while applying torque to wrench flats on the tibial reamer bit and counter-torque to the flat-sided feature on the flexible reamer; and unthreading the tibial reamer bit.

30. The method of claim 29, further comprising inserting a removal tool into a hole in the tibial reamer bit to facilitate detachment from the flexible reamer.

31. The stem reamer guide of claim 12, in combination with the alignment guide of claim 1 and the alignment assembly of claim 8, wherein the stem reamer guide is configured to be inserted into a cavity opening at the distal end of the tibia after formation of the cavity with the 90-degree reamer guided by the alignment guide.
